(19) United States
(12) Patent Application Publication
(10) Pub. No.: US 2016/0367171 A1
(43) Pub. Date: Dec. 22, 2016

(54) POSTURE DETERMINATION DEVICE AND POSTURE DETERMINATION METHOD

(71) Applicant: SUMITOMO RIKO COMPANY LIMITED, Komaki-shi (JP)

(72) Inventor: Atsuki SHIMIZU, Komaki-shi (JP)

(73) Assignee: SUMITOMO RIKO COMPANY LIMITED, Komaki-shi (JP)

(21) Appl. No.: 15/254,708

(22) Filed: Sep. 1, 2016

Related U.S. Application Data
(63) Continuation of application No. PCT/JP2014/064657, filed on Jun. 2, 2014.

(51) Int. Cl. A61B 5/11; A61G 7/05; G01L 5/00; A61B 5/107

(52) U.S. Cl. CPC A61B 5/1116; A61B 5/107; A61B 5/1117; A61G 7/05; G01L 5/00

(57) ABSTRACT

A posture determination device for determining a user's posture including: a plurality of pressure detection parts disposed in a matrix arrangement on a human body support surface of bedding; a pressure sensing center calculation member to calculate a pressure sensing center of the pressure detection parts based on output values of the pressure detection parts; a determination area setting member to set at least one determination area around the calculated pressure sensing center; and a posture determination member to determine the posture based on distribution condition of the output values of the pressure detection parts within the determination area.

### INCORPORATED BY REFERENCE

This is a Continuation of International Application No. PCT/JP2014/064657 filed on Jun. 2, 2014, the disclosure of which including the specification, drawings and abstract is incorporated herein by reference in its entirety.

### BACKGROUND OF THE INVENTION

1. Field of the Invention

The present invention relates to a posture determination device and a posture determination method for determining a user's posture on bedding such as a bed or the like.

2. Description of the Related Art

Conventionally, there is known a posture determination device for determining a user's posture on bedding based on output values from a plurality of pressure detection parts disposed in a matrix arrangement on a human body support surface of bedding. Such a device is disclosed in Japanese Patent No. JP-B-3138451, for example. By using this posture determination device with a living body monitor device or the like, it is possible to accurately recognize the living body information from the living body monitor device in consideration of the user's posture as well.

Meanwhile, the posture determination device of conventional construction disclosed in JP-B-3138451 recognizes the pressurized region from the output values of the plurality of pressure detection parts, and also detects whether the user is lying with his/her body extended, with his/her legs bent, or the like from the length of the pressurized region in the lengthwise direction of the bedding. Besides, U.S. Publication No. US 2005/0107722 discloses the art that determines whether the user is either in a spine position or in a lateral position based on the degree of changes in load in the row direction, by using the output values of the load sensors.

However, whereas these conventional posture determination devices make determinations based on the output values of the pressure detection parts, both of them depend on the fixed direction of the device itself such as the lengthwise direction of the bedding or the row direction of the pressure detection parts to determine the user's posture. This may suffer from a problem that in the case where the user on the bedding lies in the direction inclined with respect to the bedding, accuracy of determining a sleeping posture becomes low.

On the other hand, as disclosed in U.S. Pat. No. 6,280,392, suggested is the art comprising the steps of: specifying the load sensors by which loads of a designated value or greater are detected; calculating a group of the load sensors adjacent to the specified load sensors as a load group; calculating a correlation function between the calculated load group and the feature models prepared in advance for every posture of the user; and determining the one with high goodness of fit as the current sleeping posture. According to this art, determination of the sleeping posture depending on the fixed direction of the device itself can be avoided. However, the step for preparing a lot of feature models of expected sleeping postures was troublesome. Moreover, since there is a limit for collectively including the sleeping posture in every direction and making a sharp distinction therebetween, it was difficult to regard it as an advantageous way for solving the problem.

### SUMMARY OF THE INVENTION

The present invention has been developed in view of the above-described matters as the background, and it is an object of the present invention to provide a posture determination device and a posture determination method which are novel and able to accurately determine the user's posture with simple means or a simple method without being influenced by the direction of the user on the bedding.

The above and/or optional objects of this invention may be attained according to at least one of the following modes of the invention. The following modes and/or elements employed in each mode of the invention may be adopted at any possible optional combinations.

A first mode of the present invention related to a posture determination device provides a posture determination device for determining a user's posture comprising: a plurality of pressure detection parts disposed in a matrix arrangement on a human body support surface of bedding; a pressure sensing center calculation member to calculate a pressure sensing center of the pressure detection parts based on output values of the pressure detection parts; a determination area setting member to set at least one determination area around the calculated pressure sensing center; and a posture determination member to determine the posture based on distribution condition of the output values of the pressure detection parts within the determination area.

With the posture determination device according to the present invention, first, based on the output values of the pressure detection parts disposed in the matrix arrangement, the pressure sensing center, which is a center part of the pressure detection parts by which the pressures are detected, is detected. Then, the determination area is set around the pressure sensing center, and the posture can be determined based on the distribution condition of the output values of the pressure detection parts within the determination area. That is, determination of the user's posture depending on the fixed direction of the device itself, as with the conventional posture determination device, is avoided and prevented. Therefore, even in the case where the user lies in an inclined state with respect to the lengthwise direction of the bedding, posture determination can be performed with the desired detection precision maintained.

Note that anything would be acceptable as the pressure sensing center as long as the center part of the pressure detection parts by which the pressures are detected can generally be specified thereby. For example, the pressure sensing center may be a center of gravity calculated by coordinate values and output values of the pressure detection parts by which the pressures are detected, or may alternatively be a center of superficial dimensions calculated by the coordinate values of the pressure detection parts by which the pressures are detected. In addition, it is acceptable as long as the determination area is set around the pressure sensing center. The number of the determination area may be one or more than one, and the size of the determination area etc. can desirably be set, depending on the determination requirements.

Moreover, the posture determination device of the present invention is able to determine the posture based on the distribution condition of the output values of the pressure detection parts within the determination area set around the pressure sensing center. Accordingly, it is possible to determine the posture in a simple way without needing complicated works such as preparing a lot of sleeping posture models in advance or calculating the correlation therebetween, as with the conventional device. Besides, despite such a simple determination method, variability in determination accuracy depending on the direction of the user on the bedding is advantageously prevented.

A second mode of the present invention related to a posture determination device provides the posture determination device according to the first mode wherein the determination area setting member sets the at least one determination area comprising a plurality of determination areas including a central area inclusive of the pressure sensing center and at least one peripheral annular area that surrounds the central area. The present mode is able to set a plurality of determination areas with the pressure sensing center arranged at the center. This makes it easy to recognize the feature of every posture in the peripheral region with the pressure sensing center arranged at the center during the posture determination of the user. Thus, the determination area settings of the present mode can advantageously enhance accuracy of the posture determination.

A third mode of the present invention related to a posture determination device provides the posture determination device according to the second mode, wherein the central area and the peripheral annular area are disposed in a concentric circular pattern. Since the central area and the peripheral annular area are disposed in a concentric circular pattern, the present mode is able to advantageously maintain accuracy of the posture determination regardless of the directions on the bedding in which the user lies.

A fourth mode of the present invention related to a posture determination device provides the posture determination device according to the second or third mode, wherein the central area and the peripheral annular area have superficial dimensions equal to each other. By setting the superficial dimensions of the central area and those of the peripheral annular area equal to each other, the present mode is able to advantageously improve accuracy of the posture determination based on the distribution condition of the output values of the pressure detection parts within the determination area.

A fifth mode of the present invention related to a posture determination device provides the posture determination device according to any one of the first through fourth modes, wherein the posture determination member comprises a grouping member to group the pressure detection parts into a plurality of groups based on the respective output values, and a number calculation member to calculate a number of the pressure detection parts that belong to each group in each determination area.

The present mode is capable of grouping the pressure detection parts into the groups based on the respective output values by the grouping member, while being capable of calculating the number of the pressure detection parts that belong to each group in each determination area by the number calculation member. This makes it possible to rapidly grasp the distribution condition of the output values of the pressure detection parts within the determination area, thereby efficiently performing the posture determination.

A sixth mode of the present invention related to a posture determination device provides the posture determination device according to the fifth mode, wherein the grouping member defines the groups with a plurality of pressure value ranges obtained by dividing a differential between a minimum output value set in advance for the pressure detection parts and a maximum output value actually measured by the pressure detection parts into a plurality of equal values. With the present mode, the groups with the pressure value ranges used for grouping are defined by dividing a differential between the minimum output value set in advance and the maximum output value among the actual measurements. Thus, appropriate grouping according to the user's weight or the like is suitably possible, and variability in posture detection precision due to conditions such as individual difference of the user can be advantageously prevented.

A seventh mode of the present invention related to a posture determination device provides the posture determination device according to the sixth mode, wherein the grouping member classifies the pressure detection parts into at least three groups, the determination area setting member sets the at least one determination area comprising a plurality of determination areas including a central area inclusive of the pressure sensing center and at least two peripheral annular areas that surround the central area in a concentric pattern, and the posture determination member includes at least one of following determination members of: a lateral-position determination member to determine the posture as a lateral position when a total number of the pressure detection parts that belong to the group with largest output values is greater than a total number of the pressure detection parts that belong to the group with smallest output values; a spine-position determination member to determine the posture as a spine position when the pressure detection parts are detected in all the groups, and a total number of the pressure detection parts that belong to the group with largest output values is smallest in comparison with each total number of the pressure detection parts that belong to other groups; a sitting-position determination member to determine the posture as a sitting position when a number of the pressure detection parts that belong to the central area is not less than 60% of a total number of the pressure detection parts that belong to all the groups; a prone-position determination member to determine the posture as a prone position when in each determination area, both of a number of the pressure detection parts that belong to the group with smallest output values and a number of the pressure detection parts that belong to the group with second smallest output values are not less than 0.875% of a total number of all the pressure detection parts disposed on the human body support surface; and an end-position determination member to determine the posture as an end position when the pressure sensing center is positioned in a column located in a range of 30% or less from an end in the matrix arrangement of the pressure detection parts.

According to the present mode, the pressure detection parts are classified into three or more groups, and the determination area is divided into the central area and two or more peripheral annular areas surrounding the central area in a concentric pattern. This makes it possible to more finely perform the posture determination of the user, thereby enhancing determination accuracy. Note that whereas the number of groups of the pressure detection parts or the total number of the determination areas can desirably be set to three or more in consideration of the disposition pitch of the pressure detection parts or the like, they may preferably be set from three to seven, more preferably be set to around five.

Also, the posture determination member may include the determination member for specific postures desirably selected as needed. Therefore, if the distribution condition of the output values of the pressure detection parts matches the above-mentioned determination condition of the lateral-position determination member, the lateral position can be determined by the lateral-position determination member. Likewise, if the distribution condition of the output values of the pressure detection parts matches the above-mentioned determination condition of the spine-position determination member, the spine position can be determined by the spine-position determination member. In addition, if the distribution condition of the output values of the pressure detection parts matches the above-mentioned determination condition of the prone-position determination member, the prone position can be determined by the prone-position determination member. By so doing, the prone position, which was difficult to identify with the conventional posture determination device, can be identified with a stable and simple system. Besides, the end-position determination member is able to efficiently detect the end position, namely, the state in which the user is close to the end of the bedding, by appropriately utilizing the pressure sensing center obtained in advance. In this way, by setting an alarm or the like to be given when the end position is detected, it is possible to prevent the user's falling down from the bed or the like before it happens. That is, with the present mode, posture determination can efficiently be performed by selecting any determination member as needed.

An eighth mode of the present invention related to a posture determination device provides the posture determination device according to the seventh mode, wherein the lateral-position determination member further comprises a left/right lateral-position sharp distinction member to make a sharp distinction between a left lateral position and a right lateral position based on a position of the pressure detection part with the maximum output value in a row direction with respect to the pressure sensing center.

According to the present mode, with respect to the posture whose distribution condition is determined as the lateral position by the lateral-position determination member, by appropriately utilizing the pressure sensing center obtained in advance, a sharp distinction can be made between the left lateral position and the right lateral position in a reliable manner, thereby more finely performing posture determination in an efficient way.

A ninth mode of the present invention related to a posture determination device provides the posture determination device according to any one of the first through eighth modes, wherein the pressure sensing center is a center of gravity obtained by the output values of the pressure detection parts by which the output values not less than a designated value are detected. By employing the center of gravity as the pressure sensing center, the present mode is able to more correctly specify the center part of the pressure detection parts by which the output values not less than the designated value are detected, thereby improving determination accuracy of the posture determination member.

A tenth mode of the present invention related to a posture determination device provides the posture determination device according to any one of the first through eighth modes, wherein the pressure sensing center is a center of superficial dimensions of the pressure detection parts by which the output values not less than a designated value are detected. By employing the center of superficial dimensions as the pressure sensing center, the present mode is able to more efficiently specify the center part of the pressure detection parts by which the output values not less than the designated value are detected.

A first mode of the present invention related to a posture determination method provides a posture determination method of determining a user's posture with a plurality of pressure detection parts disposed in a matrix arrangement on a human body support surface of bedding, the method comprising: a pressure sensing center calculation step of calculating a pressure sensing center of the pressure detection parts based on output values of the pressure detection parts; a determination area setting step of setting at least one determination area around the calculated pressure sensing center; and a posture determination step of determining the posture based on distribution condition of the output values of the pressure detection parts within the determination area.

Owing to the posture determination method according to the present invention, the same as the above-described posture determination device of the present invention, it is possible to advantageously prevent variability in determination accuracy depending on the direction of the user on the bedding and reliably make a posture determination with a simple determination method.

A second mode of the present invention related to a posture determination method provides the posture determination method according to the first mode, wherein the posture determination step comprises a grouping step of grouping the pressure detection parts into a plurality of groups based on the respective output values, and a number calculation step of calculating a number of the pressure detection parts that belong to each group in each determination area.

According to the present mode, the grouping step and the number calculation step make it possible to rapidly grasp the distribution condition of the output values of the pressure detection parts within the determination area, thereby efficiently performing the posture determination.

A third mode of the present invention related to a posture determination method provides the posture determination method according to the second mode, wherein the grouping step classifies the pressure detection parts into at least three groups, the determination area setting step sets the at least one determination area comprising a plurality of determination areas including a central area inclusive of the pressure sensing center and at least two peripheral annular areas that surround the central area in a concentric pattern, and the posture determination step includes at least one of following determination steps of: a lateral-position determination step of determining the posture as a lateral position when a total number of the pressure detection parts that belong to the group with largest output values is greater than a total number of the pressure detection parts that belong to the group with smallest output values; a spine-position determination step of determining the posture as a spine position when the pressure detection parts are detected in all the groups, and a total number of the pressure detection parts that belong to the group with largest output values is smallest in comparison with each total number of the pressure detection parts that belong to other groups; a sitting-position determination step of determining the posture as a sitting position when a number of the pressure detection parts that belong to the central area is not less than 60% of a total number of the pressure detection parts that belong to all the groups; a prone-position determination step of determining the posture as a prone position when in each determination area, both of a number of the pressure detection parts that belong to the group with smallest output values and a number of the pressure detection parts that belong to the group with second smallest output values are not less than 0.875% of a total number of all the pressure detection parts disposed on the human body support surface; and an end-position determination step of determining the posture as an end position when the pressure sensing center is positioned in a column located in a range of 30% or less from an end in the matrix arrangement of the pressure detection parts.

According to the present mode, the same as the above-described posture determination device of the present invention, it is possible to rapidly grasp the distribution condition of the output values of the pressure detection parts within the determination area. Thus, a desired posture determination will be efficiently performed by means of any posture determination step selected as needed.

A fourth mode of the present invention related to a posture determination method provides the posture determination method according to the third mode, wherein the lateral-position determination step further comprises a left/right lateral-position sharp distinction step of making a sharp distinction between a left lateral position and a right lateral position based on a position of the pressure detection part with a maximum output value in a row direction with respect to the pressure sensing center. According to the present mode, with respect to the posture whose distribution condition is determined as the lateral position by the lateral-position determination member, by appropriately utilizing the pressure sensing center obtained in advance, a sharp distinction can be made between the left lateral position and the right lateral position in a reliable manner, thereby more finely performing posture determination in an efficient way.

With the posture determination device and the posture determination method according to the present invention, the pressure sensing center, which is a center part of the plurality of pressure detection parts by which the pressures are detected, is detected, and the plurality of determination areas are set around the pressure sensing center, so as to determine the posture based on the distribution condition of the output values of the pressure detection parts within the determination areas. By so doing, it is possible to advantageously prevent variability in determination accuracy depending on the direction of the user on the bedding and reliably make a posture determination with a simple determination method.

### BRIEF DESCRIPTION OF THE DRAWINGS

The foregoing and/or other objects, features and advantages of the invention will become more apparent from the following description of a preferred embodiment with reference to the accompanying drawings in which like reference numerals designate like elements and wherein:

FIG. 1 is a perspective assembly view of a bed including a posture determination device of the present invention;

FIG. 2 is a cross-section view taken along line 2-2 of FIG. 1;

FIG. 3 is a block diagram showing a system constitution of the posture determination device of FIG. 1;

FIG. 4 is a top view of a body pressure sensor;

FIG. 5 is a cross-section view taken along line 5-5 of FIG. 4;

FIG. 6 is a flowchart showing the posture determination method of the present invention;

FIG. 7 is a flowchart showing a posture determination step in FIG. 6;

FIG. 8 is a view suitable for explaining determination areas set by a determination area setting step;

FIGS. 9A-9G are views suitable for explaining a distribution condition by which a posture is determined as a prone position;

FIGS. 10A-10G are views suitable for explaining a distribution condition by which a posture is determined as a left lateral position;

FIGS. 11A-11G are views suitable for explaining a distribution condition by which a posture is determined as a right lateral position;

FIGS. 12A-12G are views suitable for explaining a distribution condition by which a posture is determined as a sitting position;

FIGS. 13A-13G are views suitable for explaining a distribution condition by which a posture is determined as a spine position;

FIGS. 14A-14G are views suitable for explaining a distribution condition by which a posture is determined as an end position;

FIGS. 15A-15G are views suitable for explaining a distribution condition by which a posture is determined as an end sitting position; and

FIG. 16 is a flowchart showing a variation of the posture determination step in FIG. 6.

### DETAILED DESCRIPTION OF PREFERRED EMBODIMENTS

Following, we will explain a first embodiment of the present invention while referring to the drawings.

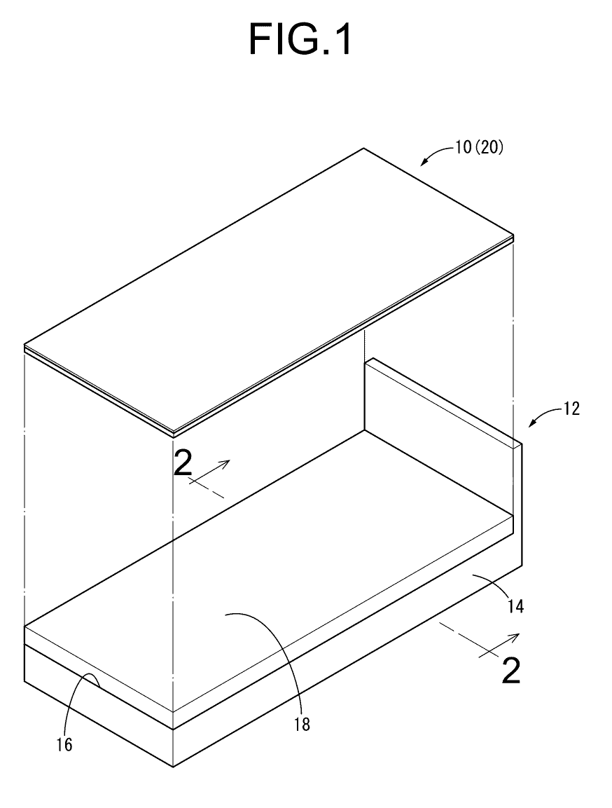

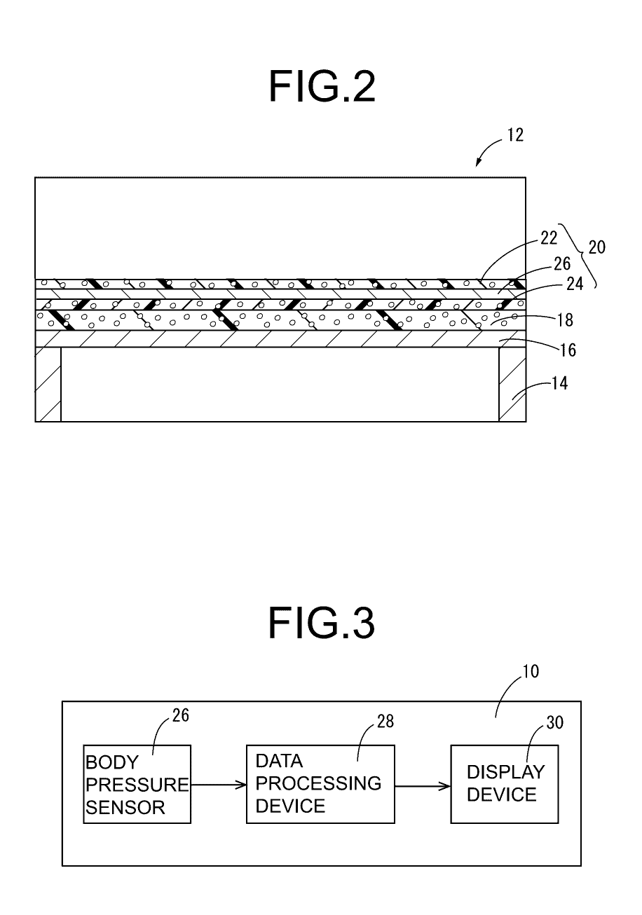

First, FIGS. 1 and 2 show a bed 12 as a bedding equipped with a posture determination device 10 constituted according to this invention. The bed 12 has a structure wherein a mattress 18 formed of a urethane foam etc. is put on the upper face of a floor plate 16 as a human body support surface in a bed main body 14. On the upper face of the mattress 18, a top mat 20 that constitutes a part of the posture determination device 10 of this invention is put. Note that, if there is no special definition in the following explanation, the longitudinal direction means the lengthwise direction of the bed 12 in FIG. 1, and the vertical direction and the lateral direction mean the up-down direction and the left-right direction in FIG. 2, respectively.

As shown in FIG. 2, the top mat 20 has substantially the same shape when vertically viewed as the mattress 18, and a rectangular plate shape thinner than the mattress 18. The top mat 20 has a layered structure including a front layer part 22 and a back layer part 24 which are each formed of a porous urethane foam. In such top mat 20, provided between the front layer part 22 and the back layer part 24 is a body pressure sensor 26 including a plurality of pressure detection parts 100 that will be described later. Although it is also possible to use a load cell etc. using a strain gauge or a magnetostrictive body as the body pressure sensor 26, a capacitance type sensor in a sheet form is used as the body pressure sensor 26 in this embodiment. For this capacitance type sensor, it is possible to use conventionally known items as appropriate.

As FIG. 3 conceptually shows, the posture determination device 10 comprises the body pressure sensor 26 housed in the top mat 20, a data processing device 28, and a display device 30. When a not-shown user lies on the top mat 20 arranged on the bed 12, the body pressure sensor 26 in the top mat 20 detects the body pressure and output signals are transmitted to the data processing device 28. Posture determination is carried out based on the output signals received by the data processing device 28, then the determination result is displayed in the display device 30.

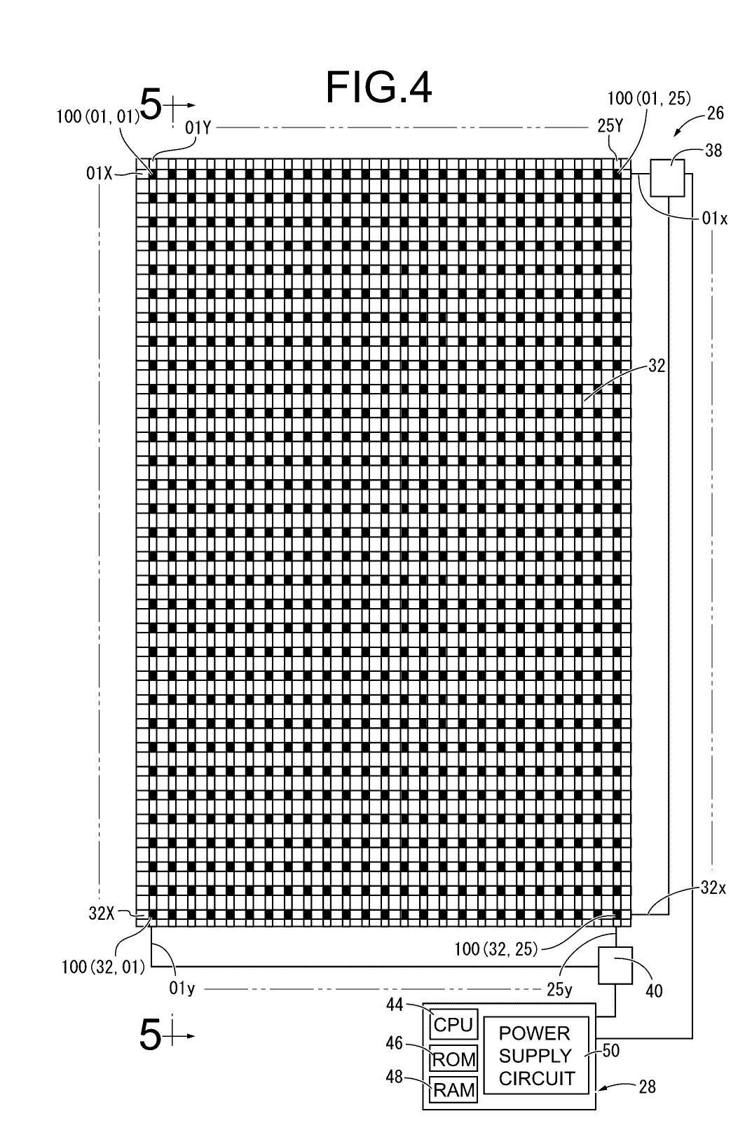

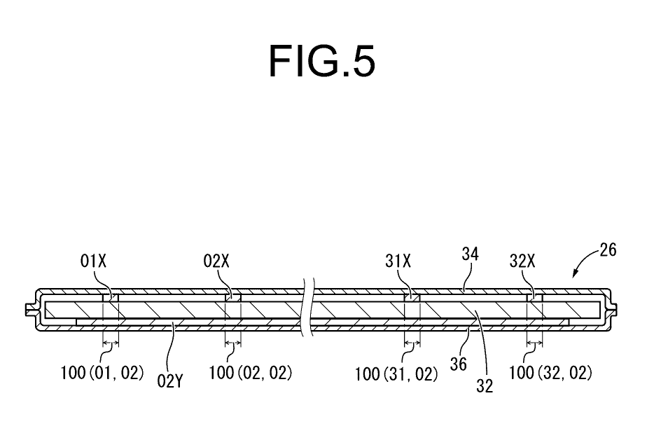

Next, the body pressure sensor 26 is schematically shown in FIGS. 4 and 5. In FIG. 4, to make it easily understandable, the body pressure sensor 26 is shown through a dielectric layer 32 and a front side base material 34 that will be described later, while the pressure detection parts 100 are shown as they are painted black.

The body pressure sensor 26 comprises the dielectric layer 32, front side electrodes 01X to 32X, back side electrodes 01Y to 25Y, front side wires 01x to 32x, back side wires 01y to 25y, the front side base material 34, a back side base material 36, a front side wiring connector 38, and a back side wiring connector 40. These front side wiring connector 38 and back side wiring connector 40 are connected electrically to the data processing device 28. Although the front side wires 01x to 32x, the back side wires 01y to 25y, the front side wiring connector 38 and the back side wiring connector 40 are all arranged in the body pressure sensor 26, they are schematically shown outside the body pressure sensor 26 in FIG. 4, in order to make them easily seeable.

The dielectric layer 32, which is made using a urethane foam body as an elastomer, is a sheet whose shape is a rectangular plate and can be subject to elastic deformation. The dielectric layer 32 is roughly equal to the upper face of the mattress 18 in size.

The front side base material 34 is made of a rubber and has a rectangular plate shape. The front side base material 34 is layered on the upper side (front side) of the dielectric layer 32. The back side base material 36 is made of a rubber and has a rectangular plate shape. The back side base material 36 is layered on the lower side (back side) of the dielectric layer 32.

As shown in FIG. 5, the outer rim of the front side base material 34 and the outer rim of the back side base material 36 are connected such that the front side base material 34 and the back side base material 36 are bonded to each other so as to form a bag. The dielectric layer 32 is housed in the bag. The four corners of the upper face of the dielectric layer 32 are spot-adhered to the four corners of the lower face of the front side base material 34. Also, the four corners of the lower face of the dielectric layer 32 are spot-adhered to the four corners of the upper face of the back side base material 36. Thus, the dielectric layer 32 is positioned in relation to the front side base material 34 and the back side base material 36 in order to avoid a wrinkle in use. However, the dielectric layer 32, with its four corners bonded, can undergo elastic deformation in the horizontal direction (front-back and left-right directions) relative to the front side base material 34 and the back side base material 36.

A total of 32 front side electrodes 01X to 32X are arranged on the upper face of the dielectric layer 32. The front side electrodes 01X to 32X are each formed including an acrylic rubber and conductive carbon black. The front side electrodes 01X to 32X each exhibit a belt shape, and are formed to be able to expand and contract flexibly. The front side electrodes 01X to 32X each extend in the lateral direction (left-right direction in FIG. 4). The front side electrodes 01X to 32X are arranged separate at prescribed intervals in the longitudinal direction (up-down direction in FIG. 4) and roughly parallel to each other.

A total of 32 front side wires 01x to 32x are arranged on the upper face of the dielectric layer 32. The front side wires 01x to 32x are each formed including an acrylic rubber and silver powder. The front side wires 01x to 32x each exhibit a linear shape. The front side wiring connector 38 is arranged at a corner of the front side base material 34 and the back side base material 36. The front side wires 01x to 32x each connect the respective end parts of the front side electrodes 01X to 32X to the front side wiring connector 38.

A total of 25 back side electrodes 01Y to 25Y are arranged on the lower face of the dielectric layer 32. The back side electrodes 01Y to 25Y are each formed including an acrylic rubber and conductive carbon black. The back side electrodes 01Y to 25Y each exhibit a belt shape, and are formed to be able to expand and contract flexibly. The back side electrodes 01Y to 25Y each extend in the longitudinal direction (up-down direction in FIG. 4). The back side electrodes 01Y to 25Y are arranged separate at prescribed intervals in the lateral direction (left-right direction in FIG. 4) and roughly parallel to each other. Thus, the front side electrodes 01X to 32X and the back side electrodes 01Y to 25Y are disposed in a matrix arrangement wherein they are orthogonal to each other as viewed from above or below.

A total of 25 back side wires 01y to 25y are arranged on the lower face of the dielectric layer 32. The back side wires 01y to 25y are each formed including an acrylic rubber and silver powder. The back side wires 01y to 25y each exhibit a linear shape. The back side wiring connector 40 is arranged at a corner of the front side base material 34 and the back side base material 36. The back side wires 01y to 25y each connect the respective end parts of the back side electrodes 01Y to 25Y to the back side wiring connector 40.

The plurality of pressure detection parts 100 provided for the body pressure sensor 26, as shown with black-painted rectangles in FIG. 4, are arranged at the parts where the front side electrodes 01X to 32X and the back side electrodes 01Y to 25Y intersect in the vertical direction (overlapped parts), so that they are arranged, across the substantially whole face of the dielectric layer 32, roughly at regular intervals in the longitudinal direction and the lateral direction. Each pressure detection part 100 includes a part of the front side electrodes 01X to 32X, a part of the back side electrodes 01Y to 25Y and a part of the dielectric layer 32. The total number of arranged pressure detection parts 100 is 800 (equal to 32 multiplied by 25). In the posture determination method performed in the posture determination device 10, which will be described later, the front side electrodes 01X to 32X and the back side electrodes 01Y to 25Y are used as x-coordinate values and y-coordinate values, respectively, so as to recognize each pressure detection part 100 as the pressure detection part 100 (x, y). For example, the pressure detection part 100 positioned at left and top end in FIG. 4, which is arranged at the intersection part of the front side electrode 01X and the back side electrode 01Y, is recognized as the pressure detection part 100 (01, 01). As well, the detection part 100 positioned at right and bottom end in FIG. 4, which is arranged at the intersection part of the front side electrode 32X and the back side electrode 25Y, is recognized as the detection part 100 (32, 25).

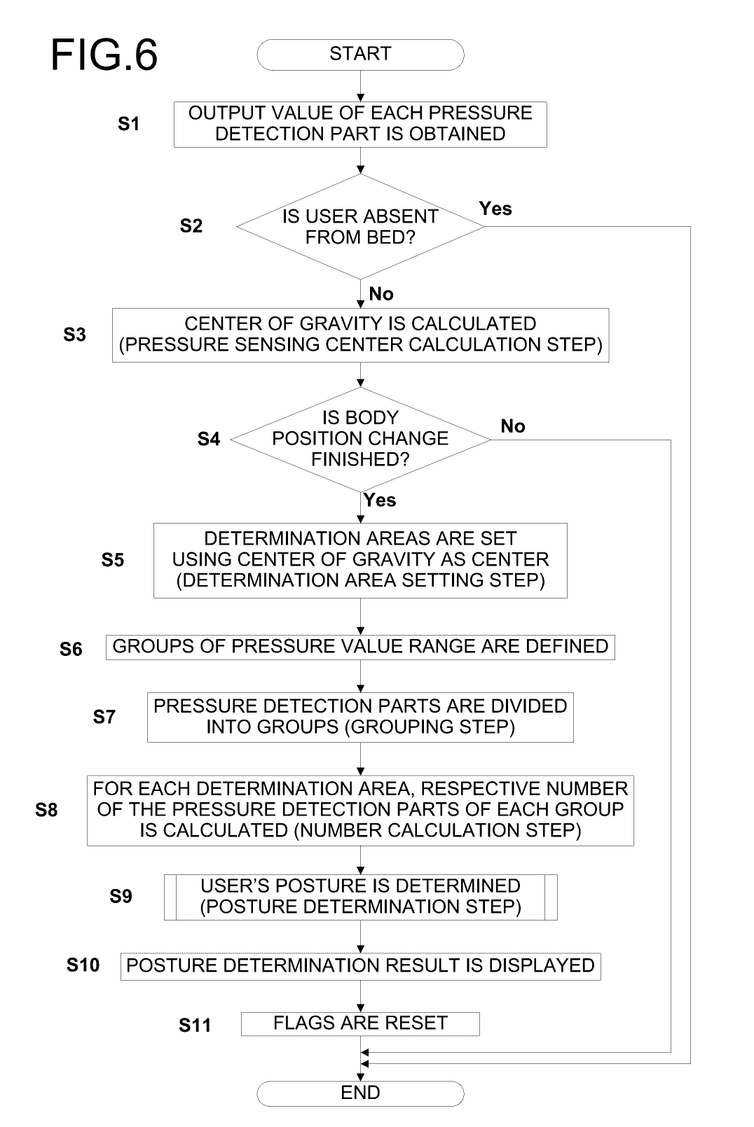

As shown in FIG. 4, the data processing device 28 comprises a CPU (Central Processing Unit) 44, a ROM (Read Only Memory) 46, a RAM (Random Access Memory) 48, and a power supply circuit 50. The ROM 46 memorizes a determination program shown in FIGS. 6, 7 and 16 based on the later-described posture determination method, a map that shows the correspondence between the electrostatic capacity of the capacitor constituted by the pressure detection parts 100 and the body pressure (load), and the like. The RAM 48 temporarily stores determination program calculation values and output values as the electrostatic capacity of the detection parts 100 input from the front side wiring connector 38 and the back side wiring connector 40. In addition, the power supply circuit 50 applies a periodical rectangular wave voltage to the pressure detection parts 100 in sequence by scanning. Then, the CPU 44 detects the body pressure acting on the pressure detection parts 100 from the electrostatic capacity of the pressure detection parts 100 memorized by the ROM 46, on the basis of the map memorized by the ROM 46. Moreover, by sending the determination result of calculation based on the determination program to the display device 30, the determination result on the basis of the posture determination method is shown by the display device 30.

The posture determination device 10 with this structure is overlapped on the floor plate 16 of the bed main body 14 as shown in FIG. 1. When the user lies on the top mat 20, the body pressure of the user acts on the top mat 20 and the mattress 18, and the user's body is supported on the floor plate 16 of the bed main body 14 that constitutes the human body support surface. As a result, on the plurality of pressure detection parts 100 disposed in a matrix arrangement in the top mat 20 on the floor plate 16 (human body support surface), the body load (body pressure) on the basis of gravity acting on the user is applied.

Following is the explanation about the first embodiment with regard to the posture determination method for determining the posture of the user on the bed 12 with the plurality of pressure detection parts 100 of the posture determination device 10. FIG. 6 shows the processing contents to be carried out in the data processing device 28 of the posture determination device 10. This process is performed repeatedly every prescribed interval of around 0.05 to 1 second, for example.

The CPU 44 of the data processing device 28 first obtains the output values as load signals output by the total of 800 pressure detection parts 100 of the body pressure sensor 26 in S1. After that, in S2, the CPU 44 checks whether the user is absent from the top mat 20 or present on the top mat 20. Specifically, the CPU 44 determines whether or not the number of pressure detection parts 100 for which it is found that the output value of each pressure detection part 100 obtained in S1 is beyond a contact threshold memorized by the ROM in advance (contact threshold<output value) is less than 0.5% of the total number of all pressure detection parts 100. In the case of less than 0.5%, it is determined that the user is absent (YES), and the following steps of the posture determination process are skipped to finish this process. In the case of not less than 0.5%, it is determined that the user is present on the top mat 20 (NO), and the process enters a step S3. Note that the contact threshold is a value by which it can be found that something gets in contact with the pressure detection part 100 significantly, and a value that can be set at will to discern the pressure detection parts 100 to be significantly used for posture determination from the others. For example, the contact threshold is set to 20.0 mmHg in the present embodiment.

In S3, the CPU 44 performs the pressure sensing center calculation step. Here, the pressure sensing center is acceptable provided that the center part of the plurality of pressure detection parts 100 in a pressure-sensing state wherein a pressure is detected can be determined by the pressure sensing center. In the present embodiment, as the pressure sensing center, the center of gravity of the plurality of pressure detection parts 100 having output values not less than the contact threshold is calculated. Specifically, using the output value of each pressure detection part 100 obtained in S1, the position of the center of gravity (Cpx, Cpy) is calculated on the basis of the following formula as a coordinate value of the pressure detection part 100 (x, y) to be memorized in the RAM 48. In the following formula, if an arbitrary pressure detection part 100 (x, y) is expressed as i, the output value of the pressure detection part 100 is expressed as pi, the x-coordinate value thereof is expressed as xi, and the y-coordinate value thereof is expressed as yi. Also, the total number of all pressure detection parts 100 (x, y) is expressed as N, and the contact threshold is expressed as t.

$$Cpx = \frac{{\sum }_{i = 1}^{N} ( {p}_{i} \times {x}_{i} )}{{\sum }_{i = 1}^{N} {p}_{i}} , Cpy = \frac{{\sum }_{i = 1}^{N} ( {p}_{i} \times {y}_{i} )}{{\sum }_{i = 1}^{N} {p}_{i}} \qquad [ Expression 1 ]$$

Note that when pi<t, pi=0

As apparent from the above description, in the present embodiment, the pressure sensing center calculation member in the posture determination device 10 comprises the CPU 44, the ROM 46 and the RAM 48 of the data processing device 28, and S3.

In S4, the CPU 44 determines whether the user on the top mat 20 is changing his/her body position or he/she is resting after changing his/her body position. Specifically, among the center-of-gravity positions which are obtained by sampling in the last three seconds memorized in the RAM 48, selected are two center-of-gravity positions which have the largest difference between their coordinate values (separate with the largest distance) so as to compare the coordinate values (x, y) of the two centers of gravity with each other. If the difference is not greater than 1 with both x and y coordinate values, it is determined that the center of gravity is not moving and the body position change has been already finished (YES), so that the process enters the posture determination steps of S5 and the following. On the other hand, if the difference is greater than 1 with at least one of x-coordinate value and y-coordinate value, it is determined that the center of gravity is moving in body position change (NO), so that the posture determination steps of S5 and the following are skipped and this process is completed.

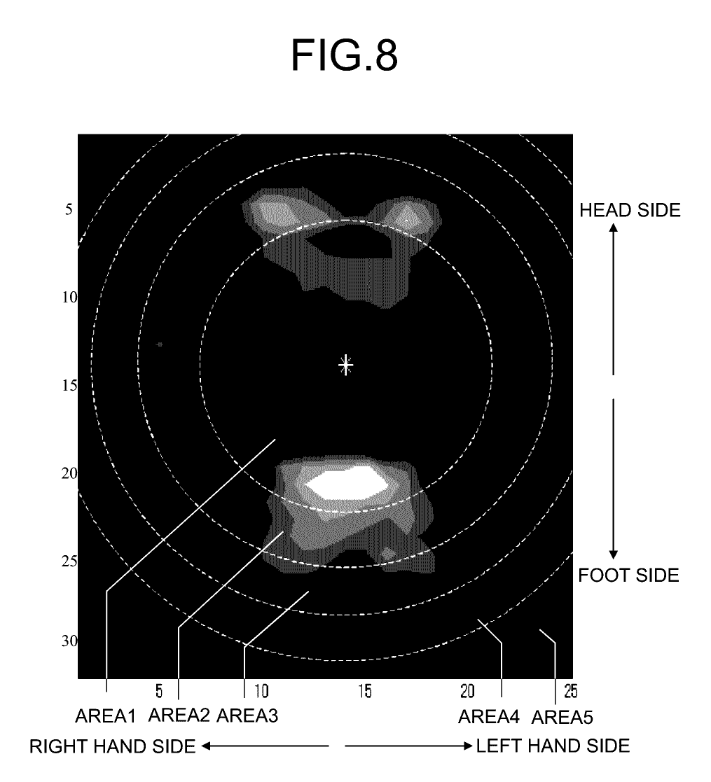

In S5, the CPU 44 performs the determination area setting step. In the present embodiment, as shown in FIG. 8, concentric circles are formed first with the center of gravity obtained in S4 (* in FIG. 8) as their center to include the center of gravity. The respective radii of the concentric circles are α, α*1.414, α*1.743, α*2 (α is a constant). Set as a result are five determination areas comprising a central area 1 defined by the center circle, peripheral annular areas 2, 3 and 4 defined in order by the concentric circles around the central area 1, and an area 5 around the peripheral annular area 4. Especially in the present embodiment, the central area 1 and the peripheral annular areas 2 to 4 have superficial dimensions equal to each other. Note that the radius α can be set at will considering the size of the entire body pressure sensor 26, the disposition pitch of the pressure detection parts 100, or the like. In the present embodiment, the radius α is 6 in coordinate value of the pressure detection part 100. In the following explanation, the central area 1 and the peripheral annular areas 2, 3 and 4 are suitably addressed as areas 1, 2, 3 and 4.

As apparent from the above-referenced description, in the present embodiment, the CPU 44, the ROM 46 and the RAM 48 of the data processing device 28, and S5 constitute the determination area setting member in the posture determination device 10.

In the following S6, the CPU 44 defines groups of pressure value range which are to be used in the grouping step of S7 described later. Specifically, the CPU 44 divides a differential between a contact threshold memorized in the ROM 46 in advance as the minimum output value of the pressure detection part 100 and the maximum output value among actually measured values of the pressure detection parts 100 obtained in S1 into five equal values. Thus, the CPU 44 obtains the plurality of groups of pressure value range to define them as a group 1, a group 2, a group 3, a group 4, and a group 5 in ascending order from a group with the smallest values. In this embodiment, the groups of pressure value range are set by dividing the differential between the contact threshold and the maximum output value in this way, thereby enabling appropriate grouping of pressure value range depending on the user's weight etc. Hence, the problem of variation in posture detection accuracy of the posture determination device 10 by a difference in the user's weight etc. is advantageously avoided.

In the following S7, the CPU 44 performs the grouping step of grouping the plurality of pressure detection parts 100 into groups 1 to 5 of pressure value range defined in S6, based on the respective output values of all the pressure detection parts 100 obtained in S1. Moreover, in the following S8, the CPU 44 performs the number calculation step of calculating the respective number of the pressure detection parts 100 belonging to each of groups 1 to 5 in each of determination areas 1 to 5 which are set in S6. In short, in the present embodiment, the grouping member and the number calculation member in the posture determination device 10 comprise the CPU 44, the ROM 46 and the RAM 48 of the data processing device 28, and S7 and S8.

In the following S9, the CPU 44 performs the posture determination step of determining the posture of the user on the top mat 20. Specifically, in the present embodiment, the posture determination member in the posture determination device 10 comprises the CPU 44, the ROM 46 and the RAM 48 of the data processing device 28, and S5 to S9.

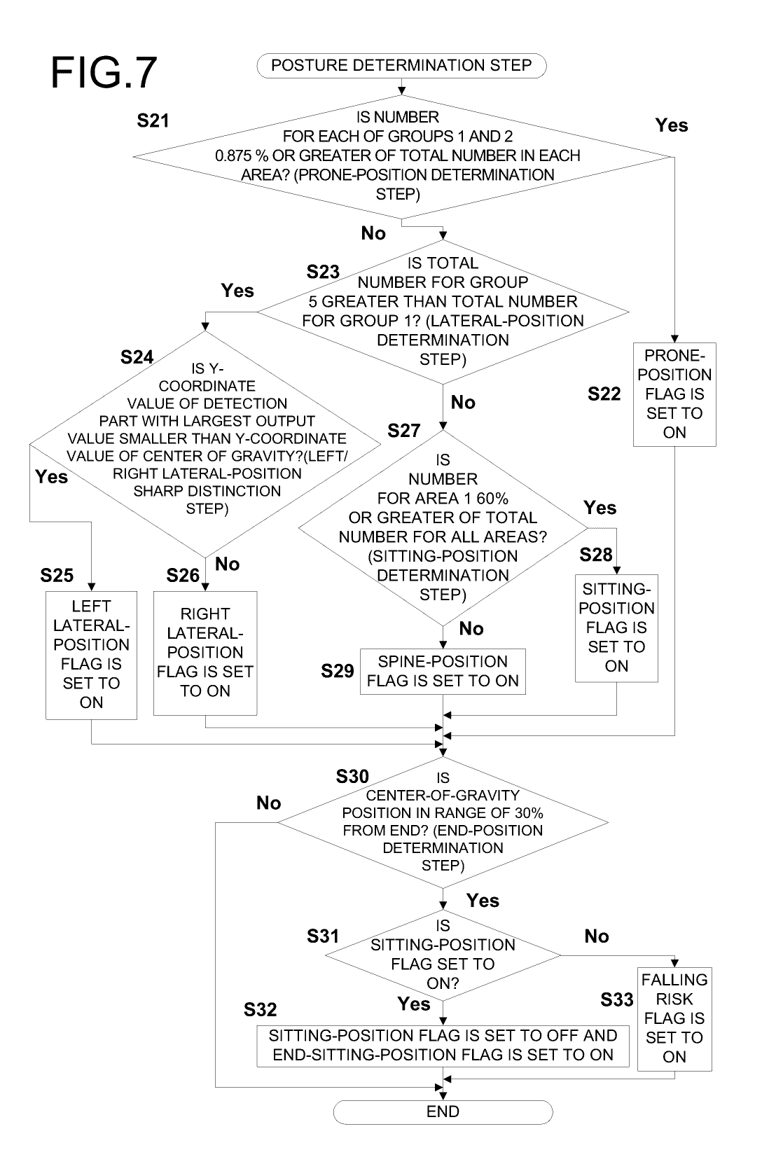

In more details, this posture determination step is performed according to the processing contents shown in FIG. 7 memorized in the ROM 46. First, the CPU 44 performs a prone-position determination step in S21. Specifically, it is determined whether or not both the number of the pressure detection parts 100 belonging to the group 1 with the smallest output values and the number of the pressure detection parts 100 belonging to the group 2 with the second smallest output values are 0.875% or greater (seven or greater in this embodiment) of the total number of all the pressure detection parts 100 disposed on the floor plate 16 of the bed 12 (800 in this embodiment), in all the determination areas 1 to 5. If the numbers for both groups are 0.875% or greater of the total number (YES), the CPU 44 sets a prone-position flag to on in S22 to proceed to S30 described later. If the number for each group is less than 0.875% of the total number (NO), the CPU 44 advances to S23.

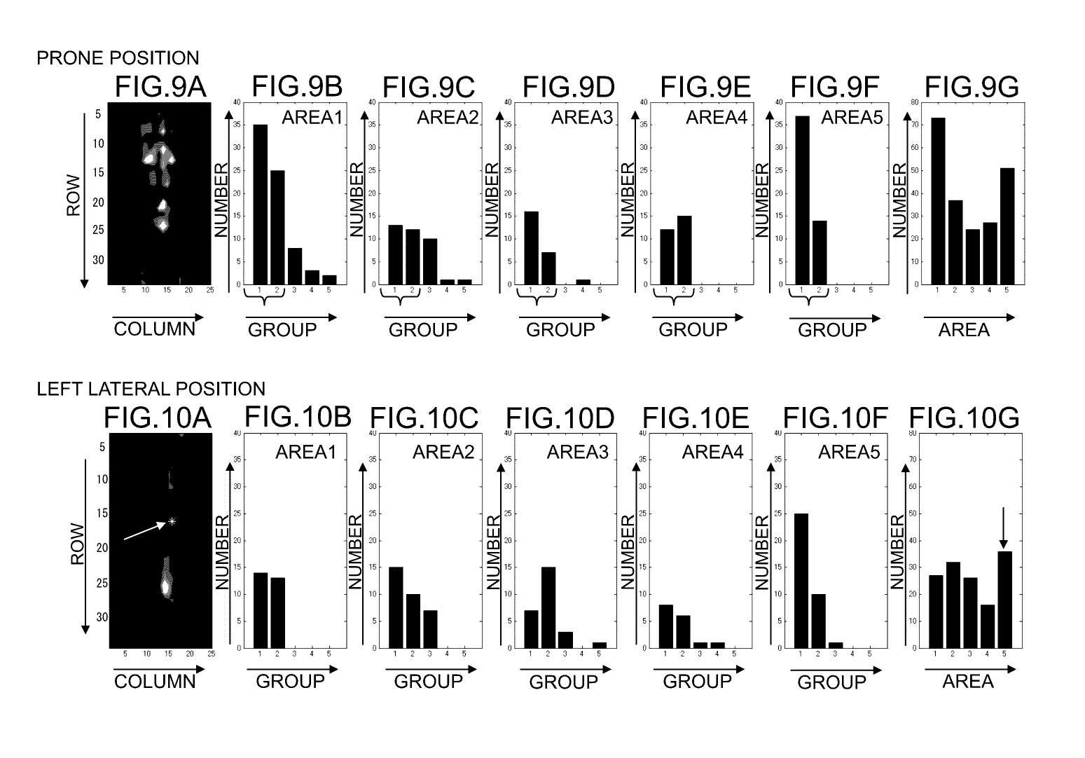

FIG. 9A shows the pressure distribution view when the user is actually lying on the top mat 20 in a prone posture. In this view, the lower the output values of the pressure detection parts 100 are, the darker the pressure detection parts 100 are displayed, while the higher the output values are, the brighter the pressure detection parts 100 are displayed. The longitudinal axis indicates the x-coordinate values of the pressure detection parts 100, while the transverse axis indicates y-coordinate values in FIG. 9A. In addition, FIGS. 9B to 9F express the pressure distribution state shown in FIG. 9A for each of the determination areas 1 to 5, in the form of bar graphs as the numbers of the pressure detection parts 100 belonging to the groups 1 to 5. In the drawings, the longitudinal axis indicates the number and the transverse axis indicates the group number. Besides, FIG. 9G expresses the pressure distribution state shown in FIG. 9A separately for each determination areas 1 to 5 as the total number of the pressure detection parts 100 belonging to any groups 1 to 5. In the drawing, the longitudinal axis indicates the number, and the transverse axis indicates the determination area number. As apparent from FIGS. 9A to 9G in the case where the user lies on the top mat 20 in a prone posture, in all the determination areas 1 to 5, the respective number of the pressure detection parts 100 belonging to each the group 1 and the group 2 is not less than 0.875% of the total number (not less than seven in this embodiment), and it can be found to meet the above-described determination condition of S22.

As apparent from the aforesaid explanation, in this embodiment, the prone-position determination member included in the posture determination member comprises the CPU 44, the ROM 46 and the RAM 48 of the data processing device 28, and S21 and S22.

Next, in S23, the CPU 44 performs a lateral-position determination step. Specifically, it is determined whether or not the total number of the pressure detection parts 100 that belong to the group 5 with the largest output values is greater than the total number of the pressure detection parts 100 that belong to the group 1 with the smallest output values. If the total number for the group 5 is greater than the total number for the group 1 (YES), the CPU 44 proceeds to S24. In S24, for the situation wherein the lateral-position determination condition is fulfilled, a left/right lateral-position sharp distinction step for making a sharp distinction between the left lateral position and the right lateral position is performed. Specifically, it is determined whether the y-coordinate value of the pressure detection part 100 with the largest output value is smaller or larger than the y-coordinate value of the center of gravity calculated in S4. If it is smaller (YES), the CPU 44 advances to S25 and sets a left lateral-position flag to on to proceed to S30. On the other hand, if the y-coordinate value of the pressure detection part 100 with the largest output value is larger than the y-coordinate value of the center of gravity (NO), the CPU 44 advances to S26 and sets a right lateral-position flag to on to proceed to S30.

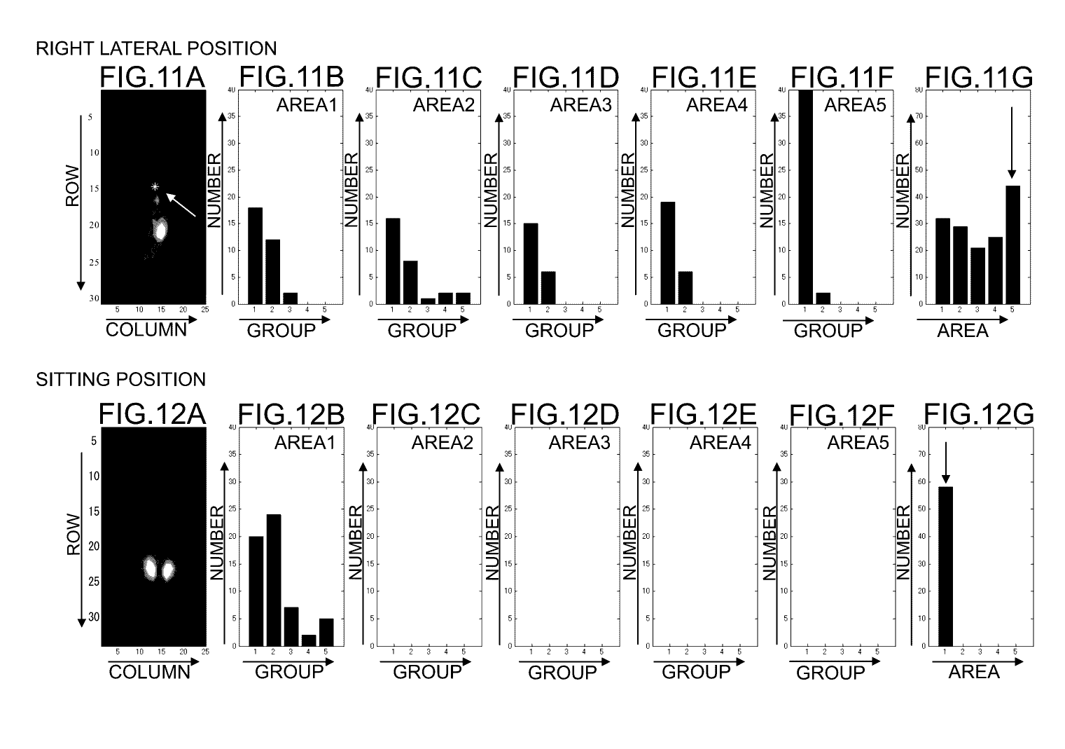

FIGS. 10A to 10G show the views corresponding to FIGS. 9A to 9G when the user actually lies on the top mat 20 in a left lateral-position posture. Also, FIGS. 11A to 11G show the views corresponding to FIGS. 9A to 9G when the user actually lies on the top mat 20 in a right lateral-position posture. As apparent from FIGS. 10A to 10G and FIGS. 11A to 11G, when the user lies on the top mat 20 in a lateral-position posture, the total number for the group 5 is greater than the total number for the group 1, and it can be found to fulfill the above-described determination condition of S23. In addition, in the case of left lateral position of FIGS. 10A to 10G the y-coordinate value of the center of gravity (*) is smaller than the y-coordinate value of the pressure detection part 100 with the largest output value. In the case of right lateral position of FIGS. 11A to 11G the y-coordinate value of the center of gravity (*) is larger than the y-coordinate value of the pressure detection part 100 with the largest output value. Therefore, it can be found that they match the determination result of S24.

As apparent from the aforesaid explanation, in this embodiment, the lateral-position determination member and the left/right lateral-position sharp distinction member included in the posture determination member comprise the CPU 44, the ROM 46 and the RAM 48 of the data processing device 28, and S23 to S26.

Next, the CPU 44 performs a sitting-position determination step in S27. Specifically, it is determined whether or not the number of the pressure detection parts 100 that belong in the area 1 as the central area is 60% or greater of the total number of the pressure detection parts 100 that belong to all the groups 1 to 5. In the case of 60% or greater (YES), the CPU 44 advances to S28 and sets a sitting-position flag to on to proceed to S30. In the case of less than 60% (NO), the CPU 44 advances to S29 and sets a spine-position flag to on to proceed to S30.

FIGS. 12A to 12G show the views corresponding to FIGS. 9A to 9G when the user is actually sitting on the top mat 20 in a sitting-position posture. As apparent from FIGS. 12A to 12G, when the user is sitting on the top mat 20 in a sitting-position posture, the number of the pressure detection parts 100 in the area 1 is 60% or greater of the total number of the pressure detection parts 100 for any groups 1 to 5, and it can be found to meet the aforementioned determination condition of S27. In the present embodiment, the sitting-position determination member included in the posture determination member comprises the CPU 44, the ROM 46 and the RAM 48 of the data processing device 28, and S27 and S28.

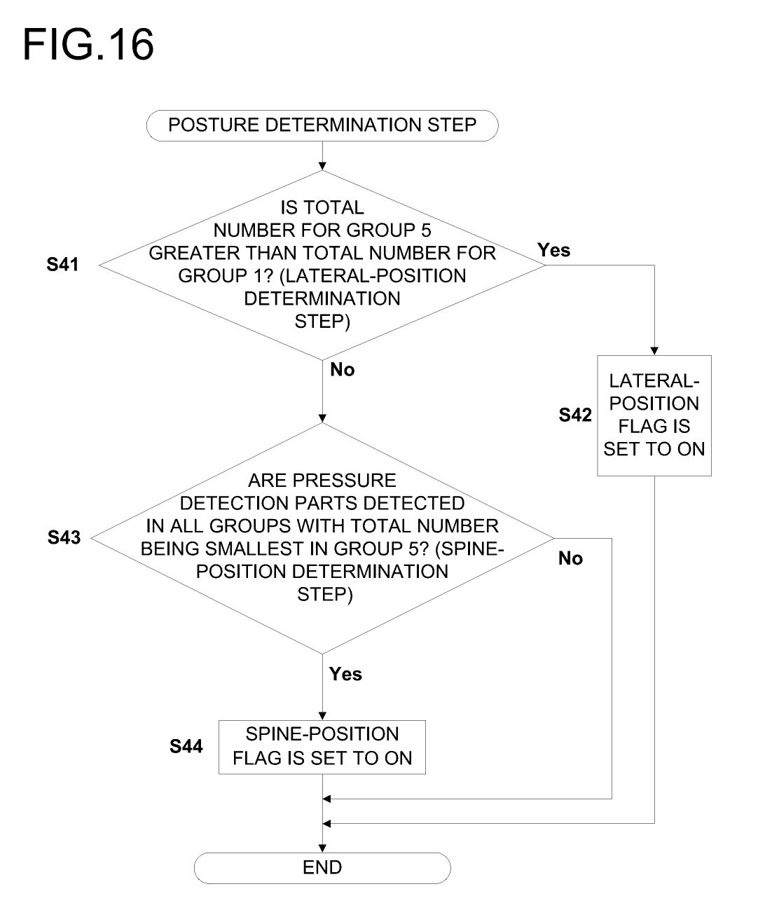

Here, in the present embodiment, the spine-position flag is set to on without performing the spine-position determination step in S29. It is because in the case wherein none of the conditions of the prone-position determination step of S21, the lateral-position determination step of S23, and the sitting-position determination step of S27 are met, a remaining posture is only the spine position, so that it is possible to determine the posture as the spine position even if the spine-position determination step is omitted. This makes the posture determination step more efficient. Note that, in S29, it is naturally possible to perform the spine-position determination step as shown in S43 in FIG. 16 described later for checking.

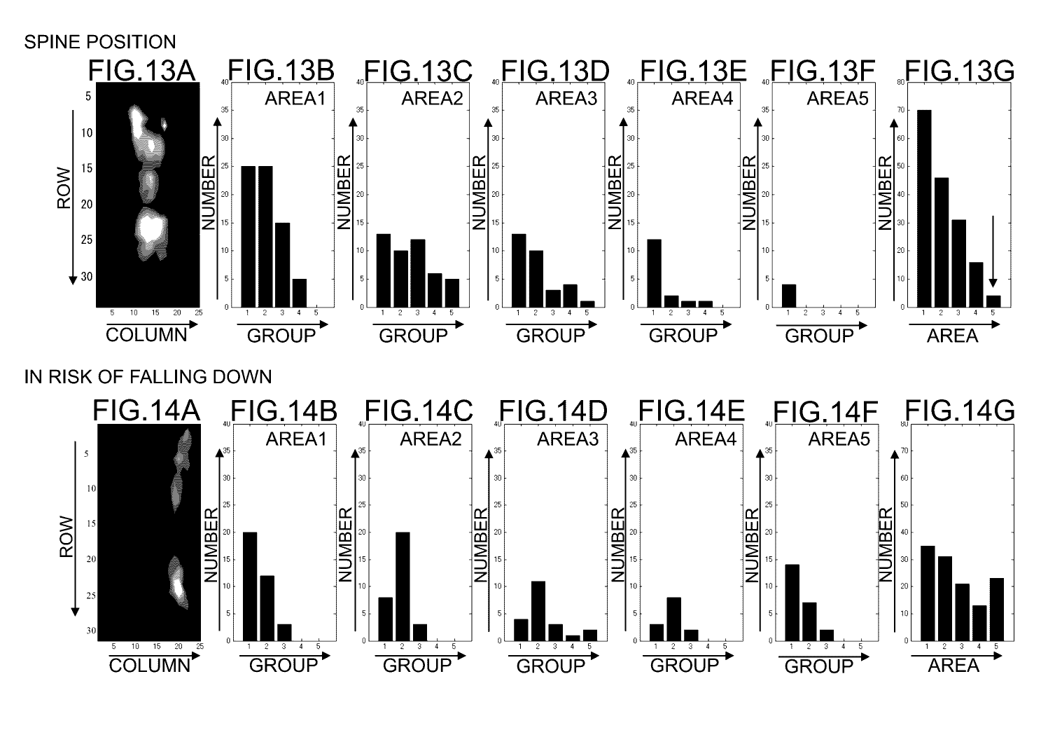

FIGS. 13A to 13G show the views corresponding to FIGS. 9A to 9G when the user is actually lying on the top mat 20 in a spine-position posture. The feature in pressure distribution for the spine position is described in a second embodiment described later.

Following, the CPU 44 performs an end-position determination step in S30. Specifically, it is determined whether or not the center of gravity calculated in S4 is positioned in a column located in a range of 30% or less from the end, in the matrix arrangement of the plurality of pressure detection parts 100. That is, if the y-coordinate value of the center of gravity is included in the y-coordinate values corresponding to the columns of within 30% from the end, it can be found that the posture is the end position for which the user is positioned on the end of the top mat 20. In the present embodiment, in the matrix arrangement of the pressure detection parts 100, the y-coordinate values are 1 to 25. Therefore, when the y-coordinate value of the center of gravity is included in either the range of 1 to 7 or the range of 19 to 25, it is determined that the posture is the end position (YES), and the process enters S31. On the other hand, when the y-coordinate value of the center of gravity is included in neither the range of 1 to 7 nor the range of 19 to 25, it is determined that the posture is not the end position (NO), and the posture determination step is finished.

If the posture is determined as the end position in S30 and the process enters S31, the CPU 44 checks whether or not the sitting-position flag is on. That is, in the case where the user is positioned on the end of the top mat 20, if the user is in the sitting position, he/she is thought to be sitting on the end of the top mat 20. In this case, it can be determined that there is no risk that the user falls down to below the bed 12 from the end of the top mat 20 in his/her sleep. Accordingly, if the sitting-position flag is on in S31 (YES), the CPU 44 sets the sitting-position flag to off and sets the end-sitting-position flag to on instead to complete the posture determination step. On the other hand, if the sitting-position flag is off in S31 (NO), the user is lying on the top mat 20 in any postures of the spine position, the prone position, the right lateral position and the left lateral position. Hence, the user can be determined to be in the risk of falling down to below the bed 12 from the end of the top mat 20. Accordingly, in this case, the CPU 44 proceeds to S33 and sets a falling risk flag to on to complete the posture determination step.

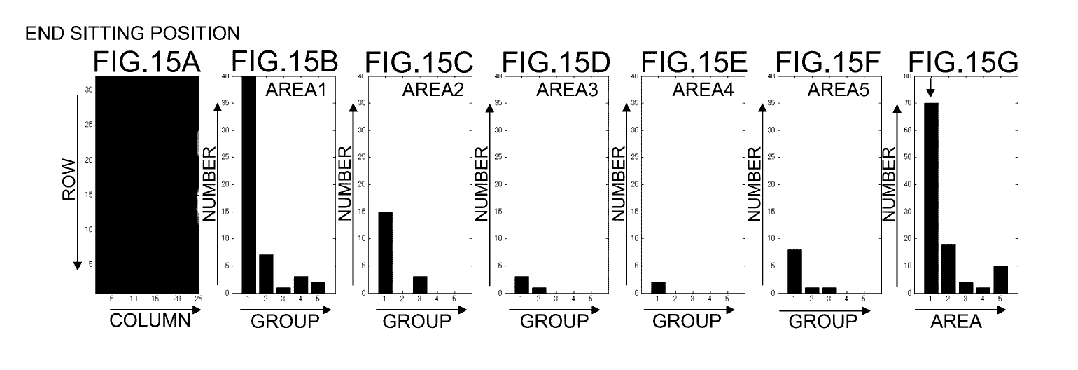

FIGS. 14A to 14G show the views corresponding to FIGS. 9A to 9G when the user is actually in a left lateral-position posture and in the end position for which the center of gravity (*) is biased toward the end of the top mat 20. In this state, in the posture determination step, the left lateral-position flag and the falling risk flag are set to on. Meanwhile, FIGS. 15A to 15G show the views corresponding to FIGS. 9A to 9G when the user is actually on the top mat 20 in the sitting-position posture and in the end position for which the center of gravity (*) is biased toward the end of the top mat 20. In this state, in the posture determination step, the end-sitting-position flag is set to on.

As apparent from the above-described explanation, in this embodiment, the end-position determination member included in the posture determination member comprises the CPU 44, the ROM 46 and the RAM 48 of the data processing device 28, and S30 to S33.

As described above, after the posture determination step is completed according to the flowchart of FIG. 7, the CPU 44 proceeds to S10 in the flowchart of FIG. 6 so as to transmit information of flags set to on, to the display device as determination result signals. This allows the display device 30 to display any one of “the spine position,” “the prone position,” “the left lateral position,” “the right lateral position,” “the sitting position” and “the end sitting position” as the posture determination result. In the case of “the spine position,” “the prone position,” “the left lateral position” or “the right lateral position,” “in the risk of falling down” may be displayed in addition. After that, the CPU 44 proceeds to S11 and resets the flags so as to complete this process.

In the posture determination device 10 and the posture determination method according to this embodiment, used is a perfectly new method which has never been in the past including the pressure sensing center calculation member (step) for detecting the center of gravity which is the pressure sensing center of the plurality of pressure detection parts 100, the determination area setting member (step) for setting the determination areas 1 to 5 around the center of gravity, and the posture determination member (step) for determining the posture on the basis of the distribution condition of the output values of the pressure detection parts 100 in these determination areas 1 to 5. Thus, unlike in the conventional posture determination device, the posture determination is never performed depending on the fixed orientation of the device itself, and even in the case where the user lies tilted to the lengthwise direction of the top mat 20, the posture determination can be performed retaining the desired detection accuracy.

In addition, it is possible to tell any of “the spine position,” “the prone position,” “the left lateral position,” “the right lateral position,” “the sitting position” and “the end sitting position” from the others, using the simple method of determining whether or not the distribution condition of the output values of the pressure detection parts 100 set around the center of gravity corresponds to the respective determination criterion for each posture. Therefore, it is possible to make a distinction for the postures more simply and clearly than the conventional posture determination method of memorizing a number of posture models prepared in advance so as to make a judgment using the correlation function of the actually measured pressure distributions and the posture models.

Especially in this embodiment, the plurality of determination areas 1 to 5 are set in the form wherein the center of gravity is arranged in the center. In the determination of the user's posture, it is easy to grasp the features for each posture in the peripheral region with the center of gravity which is the pressure sensing center arranged in the center. Therefore, by the setting of the determination areas 1 to 5 of the present embodiment, the posture determination accuracy can be enhanced more advantageously. Besides, in the present embodiment, the determination areas 1 to 5 are in a concentric circle pattern and the determination areas 1 to 4 are set to have the same superficial dimension. Accordingly, it is possible to grasp the features for each posture depending on the distribution condition of the output values of the pressure detection parts 100 with even higher precision, whichever direction the user lies on the top mat 20 in.

Additionally, it is possible to rapidly recognize the distribution condition of the output values of many pressure detection parts 100 by the grouping member (step), thereby efficiently performing the posture determination.

Moreover, the left/right lateral-position sharp distinction member (step) and the end-position determination member (step) can be performed efficiently using the center of gravity which is to be required in the determination area setting member, thereby further facilitating the device and the method and improving the efficiency thereof.

Next, in accordance with FIG. 16, the posture determination step adopted in the posture determination device and the posture determination method as the second embodiment of the present invention are illustrated. In the second embodiment, the difference from the first embodiment is only in the specific procedures of the posture determination step shown in FIG. 16, and the other features are the same as those of the first embodiment, so that the explanations thereabout are omitted.

In short, for the second embodiment, the posture determination procedures shown in FIG. 16 are performed in S9 shown in FIG. 6. The posture determination step of this embodiment takes a simple form that is for determination only about “the spine position” and “the lateral position.” First, in S41, the CPU 44 carries out the lateral-position determination step. Specifically, as well as S23 in the first embodiment, when the total number for the group 5 is greater than the total number for the group 1 (YES), the CPU 44 determines the posture as the lateral position to proceed to S42 and set the lateral-position flag to on so as to complete the posture determination step.

When the total number for the group 5 is not greater than the total number for the group 1 (NO), the CPU 44 proceeds to S43 and performs the spine-position determination step. Specifically, in S43, it is determined whether or not the pressure detection parts 100 are detected in all the groups 1 to 5 and the total number of the pressure detection parts 100 that belong to the group 5 with the largest output values is the smallest in comparison with each total number of the pressure detection parts 100 that belong to all other groups 1 to 4. If this condition is met (YES), the CPU 44 advances to S44 and sets the spine-position flag to on so as to complete the posture determination step. If this condition is not met (NO), the CPU 44 determines that the distribution condition of the output values of the pressure detection parts 100 corresponds to neither the spine position nor the lateral position, and completes the posture determination step with both flags set to off.

FIGS. 13A to 13G show the views corresponding to FIGS. 9A to 9G when the user is actually lying on the top mat 20 in a spine-position posture. As apparent from FIG. 13G it can be found that, when the user lies on the top mat 20 in a spine-position posture, the pressure detection parts 100 are detected in all the groups 1 to 5 and the total number of the pressure detection parts 100 in the group 5 is the smallest in comparison with each total number of the pressure detection parts 100 of all the groups 1 to 4, so that it fulfills the aforementioned determination condition of S43. In the present embodiment, the spine-position determination member included in the posture determination member comprises the CPU 44, the ROM 46 and the RAM 48 of the data processing device 28, and S43 and S44.

The plurality of embodiments of the posture determination device 10 and the posture determination method of the present invention are described above. However, the present invention is not limited by these specific descriptions. For example, although the center of gravity is adopted as the pressure sensing center in the aforesaid embodiment, the center of superficial dimensions calculated by the coordinate values (x, y) of the plurality of pressure detection parts 100 by which the pressure is detected can be used instead. Even in such a case, the center part of the plurality of pressure detection parts by which the pressure is detected can be determined generally, and in the posture determination, the same effect as that of the above-described embodiment is exhibited.

The center of superficial dimensions (Cax, Cay) as the pressure sensing center is calculated as the coordinate value (x, y) of the pressure detection part 100, based on the following formula. Note that, in the case where an arbitrary pressure detection part 100 (x, y) is i in the following formula, the output value of the pressure detection part 100 is expressed as pi, the x-coordinate value thereof is expressed as xi, and the y-coordinate value thereof is expressed as yi. Additionally, the total number of all the pressure detection parts 100 (x, y) is expressed as N, the contact threshold is expressed as t, and the number of the pressure detection parts 100 (x, y) having output values not less than the contact threshold is expressed as n.

$$Cax = \frac{{\sum }_{i = 1}^{N} {x}_{i} {δ}_{i}}{n} , Cay = \frac{{\sum }_{i = 1}^{N} {y}_{i} {δ}_{i}}{n} \\ Note that n = {\sum }_{i = 1}^{N} {δ}_{i} , {δ}_{i} = \{ \begin{matrix}1 & if {p}_{i} \geq t \\ 0 & else\end{matrix} \qquad [ Expression 2 ]$$

Also, the number, the size, the shape, and the like of the determination areas set in the determination area setting member (step) are not limited to those of the above-described embodiment and can be set at will depending on the contents of the posture determination, the number or the disposition form of the pressure detection parts 100 and the like. For example, it is possible to set one determination area, and it is also possible to set a plurality of determination areas. Besides, the shapes of the central area 1 and the peripheral annular areas 2 to 4 are not limited to concentric circles and the shapes can be concentric rectangles or the like. Furthermore, the superficial dimensions of the determination areas can be mutually different corresponding to the necessity, naturally.

Additionally, the posture determination member (step) is allowable as long as it includes a determination member (step) for at least one posture, and the posture determination member (step) can be constituted at will corresponding to the need. For example, the posture determination member (step) can be constituted including all of “the spine position,” “the prone position,” “the left lateral position,” “the right lateral position,” “the sitting position” and “the end position” like the first embodiment. Also, the posture determination member (step) can be constituted including only the spine-position determination and the lateral-position determination like the second embodiment. Moreover, it is also possible to constitute the posture determination member (step) including only the prone-position determination in order to avoid the risk of baby's suffocation etc., naturally.

Besides, the plurality of pressure detection parts can be provided using the pressure detection parts 100 included in the body pressure sensor 26 like this embodiment. Also, the pressure detection parts can be constituted by disposing a plurality of pressure sensors etc. independent from each other in a matrix arrangement. Furthermore, as the structure of the pressure detection parts, it is possible to use any structures including a capacitance type, a strain gauge, a load cell, and the like, provided that the contact pressure can be measured with the structure.

What is claimed is:

1. A posture determination device for determining a user's posture comprising:
a plurality of pressure detection parts disposed in a matrix arrangement on a human body support surface of bedding;
a pressure sensing center calculation member to calculate a pressure sensing center of the pressure detection parts based on output values of the pressure detection parts;
a determination area setting member to set at least one determination area around the calculated pressure sensing center; and
a posture determination member to determine the posture based on distribution condition of the output values of the pressure detection parts within the determination area.

2. The posture determination device according to claim 1, wherein the determination area setting member sets the at least one determination area comprising a plurality of determination areas including a central area inclusive of the pressure sensing center and at least one peripheral annular area that surrounds the central area.

3. The posture determination device according to claim 2, wherein the central area and the peripheral annular area are disposed in a concentric circular pattern.

4. The posture determination device according to claim 2, wherein the central area and the peripheral annular area have superficial dimensions equal to each other.

5. The posture determination device according to claim 1, wherein the posture determination member comprises a grouping member to group the pressure detection parts into a plurality of groups based on the respective output values, and a number calculation member to calculate a number of the pressure detection parts that belong to each group in each determination area.

6. The posture determination device according to claim 5, wherein the grouping member defines the groups with a plurality of pressure value ranges obtained by dividing a differential between a minimum output value set in advance for the pressure detection parts and a maximum output value actually measured by the pressure detection parts into a plurality of equal values.

7. The posture determination device according to claim 6, wherein the grouping member classifies the pressure detection parts into at least three groups,
the determination area setting member sets the at least one determination area comprising a plurality of determination areas including a central area inclusive of the pressure sensing center and at least two peripheral annular areas that surround the central area in a concentric pattern, and
the posture determination member includes at least one of following determination members of:
a lateral-position determination member to determine the posture as a lateral position when a total number of the pressure detection parts that belong to the group with largest output values is greater than a total number of the pressure detection parts that belong to the group with smallest output values;
a spine-position determination member to determine the posture as a spine position when the pressure detection parts are detected in all the groups, and a total number of the pressure detection parts that belong to the group with largest output values is smallest in comparison with each total number of the pressure detection parts that belong to other groups;
a sitting-position determination member to determine the posture as a sitting position when a number of the pressure detection parts that belong to the central area is not less than 60% of a total number of the pressure detection parts that belong to all the groups;
a prone-position determination member to determine the posture as a prone position when in each determination area, both of a number of the pressure detection parts that belong to the group with smallest output values and a number of the pressure detection parts that belong to the group with second smallest output values are not less than 0.875% of a total number of all the pressure detection parts disposed on the human body support surface; and
an end-position determination member to determine the posture as an end position when the pressure sensing center is positioned in a column located in a range of 30% or less from an end in the matrix arrangement of the pressure detection parts.

8. The posture determination device according to claim 7, wherein the lateral-position determination member further comprises a left/right lateral-position sharp distinction member to make a sharp distinction between a left lateral position and a right lateral position based on a position of the pressure detection part with the maximum output value in a row direction with respect to the pressure sensing center.

9. The posture determination device according to claim 1, wherein the pressure sensing center is a center of gravity obtained by the output values of the pressure detection parts by which the output values not less than a designated value are detected.

10. The posture determination device according to claim 1, wherein the pressure sensing center is a center of superficial dimensions of the pressure detection parts by which the output values not less than a designated value are detected.

11. A posture determination method of determining a user's posture with a plurality of pressure detection parts disposed in a matrix arrangement on a human body support surface of bedding, the method comprising:
a pressure sensing center calculation step of calculating a pressure sensing center of the pressure detection parts based on output values of the pressure detection parts;
a determination area setting step of setting at least one determination area around the calculated pressure sensing center; and
a posture determination step of determining the posture based on distribution condition of the output values of the pressure detection parts within the determination area.

12. The posture determination method according to claim 11, wherein the posture determination step comprises a grouping step of grouping the pressure detection parts into a plurality of groups based on the respective output values, and a number calculation step of calculating a number of the pressure detection parts that belong to each group in each determination area.

13. The posture determination method according to claim 12, wherein
the grouping step classifies the pressure detection parts into at least three groups,
the determination area setting step sets the at least one determination area comprising a plurality of determination areas including a central area inclusive of the pressure sensing center and at least two peripheral annular areas that surround the central area in a concentric pattern, and
the posture determination step includes at least one of following determination steps of:
a lateral-position determination step of determining the posture as a lateral position when a total number of the pressure detection parts that belong to the group with largest output values is greater than a total number of the pressure detection parts that belong to the group with smallest output values;
a spine-position determination step of determining the posture as a spine position when the pressure detection parts are detected in all the groups, and a total number of the pressure detection parts that belong to the group with largest output values is smallest in comparison with each total number of the pressure detection parts that belong to other groups;
a sitting-position determination step of determining the posture as a sitting position when a number of the pressure detection parts that belong to the central area is not less than 60% of a total number of the pressure detection parts that belong to all the groups;
a prone-position determination step of determining the posture as a prone position when in each determination area, both of a number of the pressure detection parts that belong to the group with smallest output values and a number of the pressure detection parts that belong to the group with second smallest output values are not less than 0.875% of a total number of all the pressure detection parts disposed on the human body support surface; and
an end-position determination step of determining the posture as an end position when the pressure sensing center is positioned in a column located in a range of 30% or less from an end in the matrix arrangement of the pressure detection parts.

14. The posture determination method according to claim 13, wherein the lateral-position determination step further comprises a left/right lateral-position sharp distinction step of making a sharp distinction between a left lateral position and a right lateral position based on a position of the pressure detection part with a maximum output value in a row direction with respect to the pressure sensing center.
